(19) United States
(12) Patent Application Publication
(10) Pub. No.: US 2010/0086542 A1
(43) Pub. Date: Apr. 8, 2010

(54) POTENTIATION OF ERYTHROPOIETIN (EPO) ACTION BY MEMBRANE STEROID RECEPTOR AGONISTS

(75) Inventors: Elias CASTANAS, Heraklion, Crete (GR); Marilena KAMPA, Heraklion, Crete (GR); Vassiliki PELEKANOU, Heraklion, Crete (GR)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: BIONATURE E.A. LTD., Nicosia (CY)

(21) Appl. No.: 12/516,340

(22) PCT Filed: Nov. 27, 2007
(86) PCT No.: PCT/GB2007/004537

§ 371 Date: Nov. 24, 2009

(30) Foreign Application Priority Data
Nov. 27, 2006 (GR) 20060100644

(51) Int. Cl. A61K 38/22; A61K 39/395; C12N 5/00; A61P 17/02; A61P 7/06; A61P 9/00; A61P 25/00

(52) U.S. Cl. 424/130.1; 514/8; 435/375

(57) ABSTRACT

The present invention relates to the use of membrane steroid receptor agonists as potentiators of the action of erythropoietin (EPO). The present invention also relates to the combined use of membrane steroid receptor agonists and erythropoietin to control apoptosis, proliferation, differentiation, migration and regeneration of cells, in different organs and tissues. Compositions comprising (i) a membrane steroid receptor agonist and (ii) erythropoietin are also provided, as are kits comprising (i) a membrane steroid receptor agonist and (ii) erythropoietin.

### FIELD OF THE INVENTION

The present invention relates to the use of membrane steroid receptor agonists as potentiators of the action of erythropoietin (EPO). More specifically, conjugates of steroids, e.g. macromolecular conjugates of steroids with proteins of any kind (serum albumin, antibodies, or other proteins, which do not permit the steroids to enter the cell and therefore allow them to function as agonists of the membrane receptors) or micromolecular conjugates or agents (for example catechin or epicatechin dimers), acting as agonists on the membrane steroid receptors, are used as enhancers of the action of erythropoietin in hemopoietic or extra-hemopoietic tissues. The invention claims that both membrane steroid receptor agonists and EPO, may be used conjointly in a kit or a composition, in order to decrease the administered doses of EPO and to minimize its side-effects, to control apoptosis, proliferation, differentiation, migration and regeneration of cells, in different organs and tissues.

Thus, the present invention further relates to compositions comprising (i) a membrane steroid receptor agonist and (ii) erythropoietin. The present invention further relates to kits comprising (i) a membrane steroid receptor agonist and (ii) erythropoietin.

### BACKGROUND OF THE INVENTION

Steroids are small lipophilic molecules which activate specific intracellular molecular targets called steroid receptors. Binding is followed by dimerization of the receptor, nuclear translocation, binding to specific steroid-regulatory elements of nucleic acids and subsequent modulation of steroid-regulated genes. This action, because of the implication of a number of discrete steps, takes time to be completed (usually hours, see Kumar M V, Tindall D J. Prog. Nucleic Acid Res. Mol. Biol. 1998; 59:289-306). In recent years however, a number of reports indicate that this sequence is not always respected. Indeed, steroids exert a number of effects in cells lacking classical receptors, while some effects occur in minutes, a time-lag non-compatible with the scheme of their classical nuclear action.

These findings led to the identification of steroid-binding elements in the plasma membrane, considered as new receptors (see Brann DW, et al Steroid Biochem. Mol. Biol. 1995; 52:113-33; Grazzini F et al Nature 1998; 392:209-512; Nadal A, et al FASEB J. 1998; 12:1341-8; Nemere I and Farach-Carson MC. Biochem. Biophys. Res. Commun. 1998; 248:443-9; Wehling M Annu. Rev. Physiol. 1997; 59:365-93). Through them, steroids exert short-term actions, including ion mobilization, secretion and cytoskeleton modifications. However, the nature of these membrane steroid sites remains elusive, although, recently, some reports identified membrane proteins (usually belonging to the seven transmembrane G-protein coupled superfamily) as specific steroid binding elements. On the other hand, rapid/nongenomic steroid action may also occur through classical intracellular receptors, anchored on the plasma membrane, either through post-translational modifications (e.g palmitoylation/myristoylation), or through interaction with a number of signaling molecules such as c-Src, PI3 kinase or unknown cell-type specific membrane proteins (reviewed in Beyer C, et al J Neurochem 2003; 87:545-50), or interaction with other growth factor receptors. The signaling of membrane steroid receptors includes activation of PI3K-Akt pathway, leading to cell survival and/or apoptosis, depending on the cell type and steroid examined (reviewed in Kampa M and Castanas E Mol Cell Endocrinol 2006; 246:76-82).

Until the identification of membrane steroid receptors, steroid-protein and specifically steroid-albumin conjugates had been used as carriers, to increase the immunogenicity of steroids (see Erlanger, B. et al. (1957) J. Biol. Chem. 228: 713; Erlanger, B. et al. (1959) J. Biol. Chem. 234: 1090; and Erlanger, B. F. et al. (1967) in Methods Enzymol. 1: 144). However, after the identification of membrane steroid receptors, these conjugates have retained an increased interest, as they may provide useful additional properties, through activation of steroid receptors at the membrane level. For example, in WO2004006966, it is disclosed the use of steroid-protein conjugates as specific modulators of cancer cell growth, based on the specific actions of each steroid category in cancer cells, in vitro and in vivo (see also Kampa M, et al Mol Cancer Ther 2006; 5:1342-51; Hatzoglou A, et al J Clin Endocrinol Metab 2005; 90:893-903).

EPO is a 30.4 kD glycoprotein, produced by the kidney in response to hypoxia, acting on erythroid progenitors to stimulate erythrogenesis. EPO exerts its actions through binding to a specific membrane receptor, a member of the cytokine receptor superfamily. EPOR dimerizes upon EPO binding, initiating signaling cascades, regulating cell proliferation, differentiation and survival (reviewed in Farrell F, and Lee A. Oncologist 2004; 9 Suppl 5:18-30). However, multiple extra-erythroid localization and actions of EPO and EPOR have been reported. EPO/EPOR have been discovered in neural tissue, developing heart and cancer cell lines, providing evidence for a potential autocrine/paracrine role (discussed in Lacombe C, and Mayeux P. Nephrol Dial Transplant 1999; 14 Suppl 2:22-8). EPOR is present in many nonerythroid cells, such as endothelial, neuronal and Leydig cells, myeloblasts, and megakaryocytes, and a number of cancer cell lines or tumors (see for example Pelekanou et al, 2007; Cancer Epidemiol Biomarkers Prey. 16, 2016-23).

In human therapeutics, EPO is administered to patients suffering from severe anemia, either due to renal failure or to cancer or cancer chemotherapy. Human recombinant EPO analogs, with different amino-acid and/or degree of glycosylation have been synthesized (see U.S. Pat. Nos. 4,833,092, 4,859,765, 4,853,871, 4,863,857, 5,733,761, 5,641,670, 5,688,679, 5,733,764, EP640619 and International Patents WO 93/09222, WO 94/12650, WO 95/31560, WO 90/11354, WO 91/06667, WO 91/09955, WO 99/05268, WO 99/66054, WO 99/38890, WO 99/11781, WO 98/05363). However, administration regimes of EPO differ, and present the additional difficulty of maintaining stable blood levels of erythroid cells. In this respect, different approaches have been tried, either through novel EPO conjugates (see WO 2002049673, WO 02/32957, WO 94/28024), by discovering new EPO formulas (EP 1723172, WO2006120030, KR20050027837) or by discovering different application regimens (WO 2005097167).

Additionally, EPO has been disclosed to be cardioprotective (WO2004047858) or protective in diabetic patients (WO2004019972), through modification of iron metabolism, or to be tissue-protective, increasing oxygenation (WO2004/022577) or through a direct protective and enhancing effect, as disclosed in WO 02053580, WO02080676, and US 2002/0086816 and 2003/0072737. In addition, as disclosed in WO2005070450, low-dose erythropoietin can stimulate physical mobilization, proliferation, and differentiation of endothelial precursor cells, stimulating vasculogenesis, treating diseases that are linked to a dysfunction of endothelial precursor cells. This ability of the agent has been claimed to give rise to new pharmaceutical formulations and uses of EPO.

Finally, an array of EPO-modified or EPO-like molecules have been described which enhance, restore function and viability of EPO-responsive tissues, organs or cells, including neuronal, retinal, muscle, heart and kidney (PCT/US01/49479, U.S. Ser. No. 10/188,905, 10/185,841, 10/612,665). In this respect, another class of molecules has been developed, which includes chimeric analogs of EPOR (US 2004/0214236). The above short review shows that EPO has, in addition to erythroid stimulating cell actions, a number of cell, tissue and organ protective activities, which are independent of its initially described actions.

Thus, it can be seen that EPO has many extremely useful therapeutic applications and the compositions and kits of the present invention provide means by which the actions of EPO can be advantageously potentiated, prolonged or increased.

In addition however, some recent reports have raised concerns with regard to the therapeutic use of EPO to treat anemia in cancer patients, as EPO might act in vivo to stimulate the growth of some tumor cells. In light of these concerns, the FDA has issued guidance instructions to practitioners that as low a dose of EPO as possible should be used in such patients, whilst still providing a sufficient dose to treat anemia (Steinbrook, R., 2007, N Engl J Med 356, 2448-2451; Fadlo R. Khuri, F. R., 2007, N Engl J Med 356, 2445-2448). The present invention provides a solution to this potential problem with EPO therapy and provides a way of meeting the FDA guidelines, by using membrane steroid receptor agonists in combination with EPO and as potentiators of the action of EPO. This use of membrane steroid receptor agonists should allow reduced doses of EPO to be used to achieve the same therapeutic effects (for example erythropoiesis), thereby minimizing the side effects of EPO administration, and in particular the side effects associated with high doses of EPO. Side effects of EPO administration might include the stimulation of the growth of tumor cells, as discussed above, but also might include conventional side effects such as thromboembolic events, cardiovascular events and increased risk of death.

### DESCRIPTION OF THE INVENTION

The present invention describes the surprising discovery of potentiation of action of erythropoietin on cell survival/apoptosis, growth, differentiation, regeneration and migration by the concomitant application of membrane steroid receptor agonists.

In a first embodiment of the present invention, the inventors describe the anti-apoptotic action of erythropoietin in non-erythroidal cells.

In a second embodiment, the inventors show the pro- or anti-apoptotic/survival action of membrane receptor agonists on non-erythroid cells. This effect depends on the cell and steroid used.

In a further embodiment, the inventors describe the surprising discovery of the potentiation of EPO effect, by the addition of estrogen or androgen membrane receptor agonists in cells completely different from erythroidal ones. This effect includes cell growth and induction or inhibition of apoptosis.

Thus, at its broadest, the present invention provides a composition comprising;
(i) a membrane steroid receptor agonist and (ii) erythropoietin (EPO).

Optionally said, compositions further comprise a pharmaceutically acceptable carrier or diluent.

The term “erythropoietin” as used herein refers to any substance with agonistic properties on EPOR. It includes native erythropoietin, recombinant or synthetic forms of erythropoietin, in particular human recombinant erythropoietin, EPOR-stimulating antibodies, erythropoietin-like peptides, functional fragments of EPO, conjugated or free EPO, EPO which contains amino-acid substitutions and/or EPO with different or altered degrees of glycosylation or sialic acid. Such substituted EPO molecules or molecules with different degrees of glycosylation or sialic acid are well known and described in the art (e.g. in U.S. Pat. Nos. 4,833,092, 4,859,765, 4,853,871, 4,863,857, 5,733,761, 5,641,670, 5,688,679, 5,733,764, EP640619 and International Patents WO 93/09222, WO 94/12650, WO 95/31560, WO 90/11354, WO 91/06667, WO 91/09955, WO 99/05268, WO 99/66054, WO 99/38890, WO 99/11781, WO 98/05363) and any of these may be used. This term also includes any form of EPO used in therapy, in particular those forms when described by their trade or manufactured names such as Epoetin alfa (marketed as Epogen, Eprex, Procrit) or Darbepoetin (marketed as Aranesp). Any person trained in the art can expand the above list to any other physical, semi-synthetic or synthetic substance with the afore-mentioned characteristics. In particular, this term thus includes EPO-modified or EPO-like molecules, as known in the art and which have an agonistic effect on EPOR.

The term “steroid” as used herein refers to any natural, semi-synthetic or synthetic molecule with agonistic activity on the appropriate homologous cognitive steroid receptor. It includes glucocorticoid, mineralocorticoid, estrogen, progestin, progesterone, androgen and vitamin-D receptor agonists. Preferred steroids are therefore glucocorticoids (e.g. cortisol), mineralocorticoids, estrogens (e.g. estradiol), progestins, progesterone, androgens (e.g. testosterone) and vitamin-D. Especially preferred steroids are estrogens or androgens, in particular estradiol and testosterone. Any person trained in the art can expand this list to any other molecule acting agonistically on the nuclear or membrane steroid receptor superfamily; for example known steroid analogs are also included.

The term “membrane steroid receptor” as used herein refers to any molecule or multi-molecular entity, loosely or tightly associated with the plasma membrane, capable of binding to free or conjugated steroids and subsequently trigger intracellular signaling cascades.

The term “membrane steroid receptor agonists” as used herein includes any molecule capable of associating, in an agonistic way, with membrane steroid receptors. It includes free and conjugated steroids, or any other natural, semi-synthetic or synthetic substance (micro- or macromolecular), deriving or not from the cholesterol skeleton, with agonistic properties on cognitive steroid receptors, membrane and/or intracellular. In particular and in addition to steroid-protein conjugates (see WO2004006966) the term extends to catechin, epicatechin and proanthocyanidolic condensed tannins, according to the document GR20050100013.

Preferred membrane steroid receptor agonists are conjugates of steroids, e.g. macromolecular conjugates of steroids with any kind of proteins. Appropriate proteins are those which do not permit the steroids to enter the cell thereby allowing the conjugate to function as an agonist of the membrane steroid receptors. Preferred examples of proteins to be used in said conjugates are mammalian proteins, preferably proteins such as globular proteins, plasma proteins, albumins or antibodies (which may be of any class, see WO2004006966). Especially preferred mammalian proteins are human serum albumin or bovine serum albumin. Micromolecular conjugates of steroids may also be included as agonists (e.g. peptide fragments might be conjugated to steroids, e.g. as described in WO2004006966). Other specific examples of micromolecular agonists are catechin, epicatechin and proanthocyanidolic condensed tannins, as described in the document GR20050100013. Further examples of membrane steroid receptor agonists are antibodies, e.g. anti membrane steroid receptor antibodies, which may be of any class and which associate in an agonistic way with membrane steroid receptors.

Exemplary membrane steroid receptor agonists are glucocorticoid, mineralocorticoid, estrogen, progestin, progesterone, androgen and vitamin-D receptor agonists. Preferred membrane steroid receptor agonists are estrogen or androgen membrane receptor agonists. Thus, preferred steroids for inclusion in the conjugates are estrogens or androgens, in particular testosterone and estradiol. In an especially preferred embodiment of the invention the steroid conjugate is a testosterone-albumin conjugate or an estradiol-albumin conjugate. Further preferred conjugates comprise human serum albumin or bovine serum albumin. In addition any natural or modified catechin, epicatechin and proanthocyanidolic condensed tannins, according to GR20050100013, may also be used.

A yet further aspect of the invention provides a product comprising (i) a membrane steroid receptor agonist and (ii) erythropoietin (EPO), as a combined preparation for simultaneous, separate or sequential administration to an animal for use to potentiate the action of EPO in hemopoietic or non-hemopoietic tissues, or for use in therapies such as those defined elsewhere herein.

A yet further aspect of the invention provides a kit comprising (i) a membrane steroid receptor agonist and (ii) erythropoietin (EPO). In preferred kits components (i) and (ii) are provided separately in a first and a second container. The kits of the invention are suitable for use and are preferably used in the methods and uses of the invention described elsewhere herein.

A yet further aspect of the invention provides the compositions, products or kits of the invention for use in therapy. Preferred therapeutic uses of the compositions of the invention are to potentiate the action of EPO, for example to enhance or restore function and viability of cells, tissues or organs, and in particular to potentiate one or more of the trophic, regenerative, proliferative and/or anti-apoptotic actions of EPO, in hemopoietic or non-hemopoietic cells, tissues or organs. Potentiation of the anti-apoptotic and/or proliferative action of EPO is an especially preferred aspect of the invention.

Various uses are also provided by the present invention. Thus, the present invention provides the use of membrane steroid receptor agonists as potentiators of the action of EPO, for example the enhancement or the restoration of function and viability of cells, tissues or organs, in particular for potentiating one or more of the trophic, regenerative, proliferative and/or anti-apoptotic action of EPO, in hemopoietic and extra-hemopoietic cells, tissues or organs. Preferably the anti-apoptotic action of EPO is potentiated.

In a further embodiment, the present invention provides the use of membrane steroid receptor agonists sequentially, simultaneously, or otherwise in addition, to erythropoietin (EPO), for the potentiation of the action of EPO, for example the enhancement or the restoration of function and viability of cells, tissues or organs, in particular for potentiating one or more of the trophic, regenerative, proliferative and/or anti-apoptotic action of EPO. Preferably the anti-apoptotic action of EPO is potentiated.

The present invention also provides the use of membrane steroid receptor agonists and EPO to control apoptosis, proliferation, differentiation, migration and/or regeneration of cells, in hemopoietic and extra-hemopoietic cells, organs and tissues. Preferably these agents are used to control apoptosis.

The present invention further provides the use of membrane receptor agonists in the manufacture of a medicament or composition to potentiate the action of EPO.

In addition, the use of membrane steroid receptor agonists and EPO in the manufacture of a medicament to control apoptosis, proliferation, differentiation, migration and/or regeneration of cells, is also provided. Preferably these agents are used to control apoptosis.

In a yet further aspect the present invention provides a method of potentiating the action of EPO, which method comprises administering to an animal an effective amount of a membrane steroid receptor agonist and EPO.

Further provided is a method of controlling apoptosis, proliferation, differentiation, migration and/or regeneration of cells, which method comprises administering to an animal an effective amount of membrane steroid receptor agonists and EPO. Preferably said method is used to control apoptosis.

The above discussed uses or methods comprise the administration of EPO and membrane steroid receptor agonists alone (e.g. simultaneously, separately or sequentially) or in combination (e.g. co-administration), in any, pharmaceutically acceptable form according to the art.

A yet further aspect of the invention provides the use of the compositions of the invention for cosmetic purposes on an animal.

The terms “potentiator”, “potentiating”, “potentiation” or “potentiate”, etc., as used herein in connection with EPO, refer to an entity which can increase or enhance the action of EPO in hemopoietic or non-hemopoietic cells, tissues or organs. Said increase or enhancement refers to a detectable or measurable increase or enhancement of action, compared to what would be seen or expected with EPO alone. Preferably the increase or enhancement is significant and more preferably statistically significant. Preferably the statistically significant increase or enhancement has a probability value of <0.1, preferably <0.05, more preferably <0.01. Appropriate methods of determining statistical significance are well known and documented in the art and any of these may be used. Preferably any such increase or enhancement is greater than additive or is synergistic (i.e. the combined effect of EPO and the membrane steroid receptor agonist is greater than the sum of their individual effects).

Any action of EPO may be potentiated by the methods and uses of the invention. However, preferred actions which are potentiated are the effects of EPO on one or more of cell survival/apoptosis, growth/proliferation, differentiation, regeneration and migration. An especially preferred action which is potentiated is the anti-apoptotic effect of EPO.

The effect of membrane steroid receptor agonists alone on cells will vary, depending on the nature of the cells, the nature of the agonist and the receptor affected. Thus, some membrane steroid receptor agonists, e.g. estradiol-albumin, have been shown to have an anti-apoptotic effect on non-erythroid cells, e.g. epithelial cells, whereas other membrane steroid receptor agonists, e.g. testosterone-albumin have been shown to have a pro-apoptotic effect on non-erythroid cells, e.g. epithelial cells. However, importantly and surprisingly, the potentiating effect of membrane steroid receptor agonists on the action of EPO, and in particular the anti-apoptotic action of EPO, has been demonstrated with membrane steroid receptor agonists which alone have either an anti-apoptotic (e.g. estradiol-albumin) or pro-apoptotic (e.g. testosterone-albumin) effect on cells. Thus, it has been demonstrated that the anti-apoptotic action of EPO is potentiated with membrane steroid receptor agonists, who alone have either pro- or anti-apoptotic activity.

Thus, a combination of an anti-apoptotic membrane steroid receptor agonist (e.g. estradiol-albumin) with EPO results in significantly reduced apoptosis compared to the reduced apoptosis seen or expected with EPO alone.

In addition, a combination of a pro-apoptotic membrane steroid receptor agonist (e.g. testosterone-albumin) with EPO results in significantly reduced apoptosis compared to the reduced apoptosis seen or expected with EPO alone.

Thus, preferred embodiments of the invention which involve the control of apoptosis provide for a reduction or a decrease in apoptosis. Such reduction or decrease in apoptosis refers to a detectable or measurable reduction or decrease in apoptosis using a membrane steroid receptor agonist and EPO in combination, compared to what would be seen or expected with either agent alone. Preferably the reduction or decrease is significant, and more preferably statistically significant. Preferably the statistically significant reduction or decrease has a probability value of <0.1, preferably <0.05, more preferably <0.01. Appropriate methods of determining statistical significance are well known and documented in the art and any of these may be used. Preferably any such reduction or decrease is greater than additive or is synergistic (i.e. the combined effect of EPO and the membrane steroid receptor agonist is greater than the sum of their individual effects).

Preferred cells (or tissues or organs comprising such cells) which are affected by the compositions, methods, etc., of the present invention, i.e. are the targets of the potentiation of EPO action, are non-erythroid cells, in particular epithelial cells, endothelial cells or mesenchymal cells. Especially preferred cells are epithelial cells. Other preferred cells are cells of bone marrow lineages, e.g. erythoid, lymphoid, mononuclear or granulocytic cells.

The in vivo methods and uses as described herein are generally carried out in animals, preferably mammals. Any mammal may be treated, for example humans and any livestock, domestic or laboratory animal. Specific examples include mice, rats, pigs, cats, dogs, sheep, rabbits, cows and monkeys. Preferably, however, the animal or mammal is a human. In vitro or ex vivo methods are however also provided.

In the manufacture of a pharmaceutical combination of the agents in accordance with the present invention or in the manufacture of a kit in accordance with the present invention, the active compounds (EPO and membrane steroid receptor agonists, including one or more pharmaceutically acceptable salts), whether present in the same composition or in separate compositions, are typically mixed with a pharmaceutically acceptable carrier. The carrier must, of course, be compatible with the active or any other ingredient in the formulation and must not be deleterious for the patient. The carrier can be solid or liquid or both and is preferably formulated with the compound as a unit-dose formulation; for example, a tablet which may contain 0.01%, 0.5%, 1%, to 99% by weight of the active compound. One or more active compounds may be incorporated in the formulation of the invention, which may be prepared by any of the techniques of pharmacy well known to those skilled in the art, consisting essentially of admixing the components, optionally including one or more accessory ingredients.

The formulation of the invention includes those suitable for external or internal use, e.g. for oral, rectal, buccal (eg sub-lingual), vaginal, parenteral (eg subcutaneous, intramuscular, intradermal, or intravenous), topical (i.e. both skin and mucosal surfaces, including airway surfaces) and transdermal administration. The most suitable route of administration will of course depend, in any particular case, on the nature and severity of the condition to be treated and the nature of the active compound. In the case of co-administration of EPO and a membrane steroid receptor agonist, parenteral administration is preferred. Other routes may be used in the case of sequential administration.

Formulations suitable for oral administration may be presented in discrete units, such as capsules, cachets, lozenges or tablets, each containing a predetermined amount of the active compound; as a powder or granules; as a solution or suspension in an aqueous or non-aqueous liquid, or in an oil-in water or water-in-oil emulsion. Such formulations may be prepared by any suitable method of pharmacy, which includes the step of bringing into association the active compound and a suitable carrier (which may contain one or more accessory ingredients, as noted above).

Formulations for parenteral administration comprise sterile aqueous and non-aqueous injection solutions of the active compound, which preferably are isotonic with the blood. The preparation may contain additionally anti-oxidants, buffers, bacteriostats and solutes rendering the solution isotonic with the blood. The formulation may be presented in uni-dose or multi-dose containers (e.g. sterile sealed ampoules and vials) and may be stored in a freeze-dried condition, requiring only the addition of the sterile liquid carrier (for example saline or water for injection), immediately before use.

For a topical use, any pharmaceutically acceptable preparation (e.g. ointment, pomade, gel, or any other pharmaceutically acceptable form known by those skilled in the art) may be used.

Preferred therapeutic applications of the compositions, kits, uses and methods of the invention are wound healing, organ, tissue and cell regeneration, cell, organ or tissue protective therapies (e.g. cardio-protective, cerebro-protective or diabetic-protective therapies), anti-apoptotic therapies or therapies in which it is desired to modulate the migration, proliferation, or differentiation of cells. Specific conditions which can be treated by the compositions, kits, uses and methods of the invention are anemia (for example anemia due to renal failure, cancer or cancer chemotherapy), heart failure, brain injury, stroke, wounds and wound healing, or any other disease, known in the art in which regenerative properties of a formulation are needed. In addition, the compositions, kits, uses and methods of the invention may be used as a primary or adjuvant appointment for cosmetic uses, in any case in which the underlying mechanism comprises tissue, organ or cell regeneration, proliferation or migration.

The terms “therapy” or “treatment” as used herein include prophylactic therapy, which may result in the prevention of disease or the delay of onset. The terms “therapy” and “treatment” include combating or cure of disease but also include the controlling, reduction or alleviation of disease or one or more of the symptoms associated therewith.

An “effective amount”, as used herein, can refer to a therapeutically effective amount or a prophylactically effective amount depending on the nature of the treatment. A therapeutically effective amount can be considered to be an amount necessary (at appropriate dosages and administration regimes) to achieve the desired therapeutic result. A prophylactically effective amount can be considered to be an amount necessary (at appropriate dosages and administration regimes) to achieve the desired prophylactic result. As indicated below, the amounts are likely to vary depending on the route of administration, weight, age and sex of the patient and the severity of the disease in the individual.

Suitable doses of the membrane steroid receptor agonists, EPO and any other active ingredients (if included) will vary from patient to patient and will also depend on the nature of the particular disease or condition to be treated. Preferably, said dosages constitute a therapeutically effective amount or a prophylactically effective amount, depending on the nature of the treatment involved. Suitable doses can be determined by the person skilled in the art or the physician in accordance with the route of administration, weight, age and sex of the patient and the severity of the disease or condition. As an example EPO administration may vary, depending on the pharmacotechnic form, from 50-1000 μg/week, preferentially 450 μg/week, parenterally. For membrane steroid receptor agonists, the preferential dose is from 1-100 mg/kg body weight for a HSA-conjugate, preferentially 5 mg/kg body weight (corresponding to a plasma concentration of 10⁻⁷M) parenterally. It should be borne in mind however that, in accordance with the present invention, the potentiating effect of the membrane steroid receptor agonists on EPO will, if desired, enable EPO to be administered at lower doses than doses required for conventional therapy, e.g. reduced by a factor of 5-10. Thus, in the case of a composition, kit, method or use of the present invention where EPO and a membrane steroid receptor agonist are used in combination, the doses of EPO, e.g. the co-administered doses of EPO, may be reduced by a factor of 5-10.

The active ingredients can be administered as a single unit dose or as multiple unit doses. In general, a weekly dose of the active agents is preferred. However, in the case of the use of micromolecular tannins as membrane steroid receptor agonists, then daily per os treatment may be used. For such a daily per os treatment an estimated daily dose per os in the range of 30-3000 μg/kg, preferably 300-500 μg/kg body weight is generally appropriate.

It is to be noted however that appropriate dosages may vary, depending on the patient and that for any particular subject, specific dosage regimes should be adjusted over time according to the individual needs of the patient. Thus, any dosage ranges set forth herein are to be regarded as exemplary and are not intended to limit the scope or practice of the claimed composition.

The compositions described herein may comprise, consist essentially of, or consist of any of the elements as described herein.

For the purpose of this specification it will be clearly understood that the word “comprising” means “including but not limited to”, and that the word “comprises” have a corresponding meaning. Therefore the words “comprise”, “comprises”, and “comprising” are to be interpreted inclusively rather than exclusively.

As used herein and in the appended claims, the singular forms “a,” “an,” and “the” include plural reference unless the context clearly dictates otherwise.

Further embodiments of the present invention are presented in the following non-limiting examples with reference to the following drawings in which:

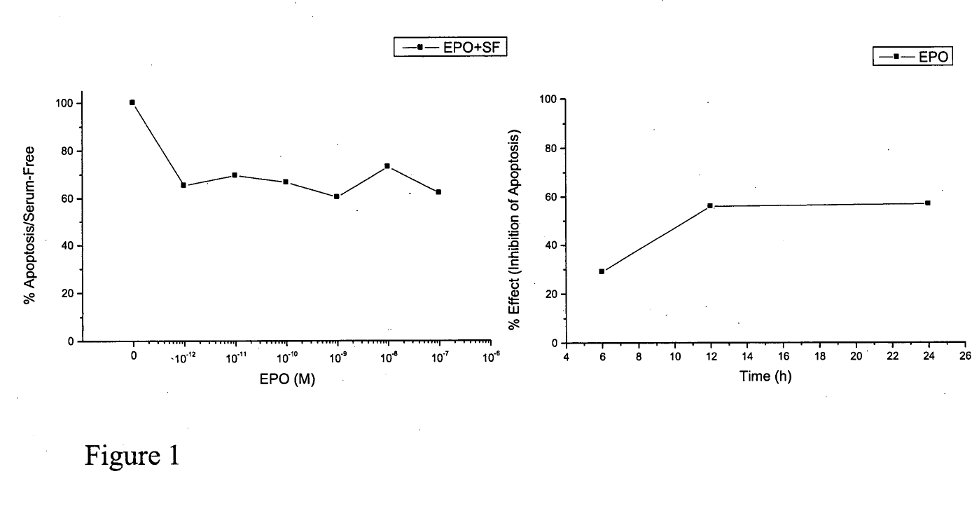

FIG. 1 presents the dose- (left panel) and time-effect of EPO on the apoptosis of T47D breast epithelial cells.

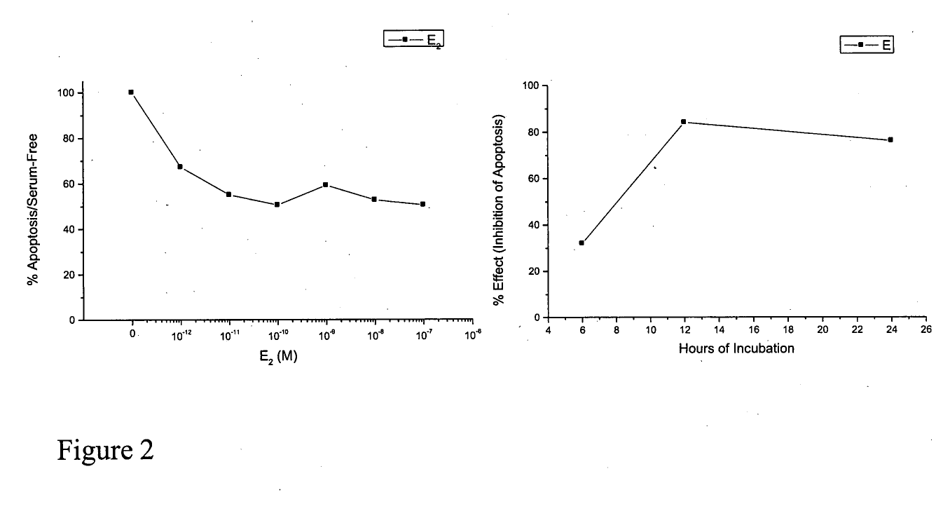

FIG. 2 presents the dose- (left panel) and time-effect of E2-BSA on the apoptosis of T47D breast epithelial cells.

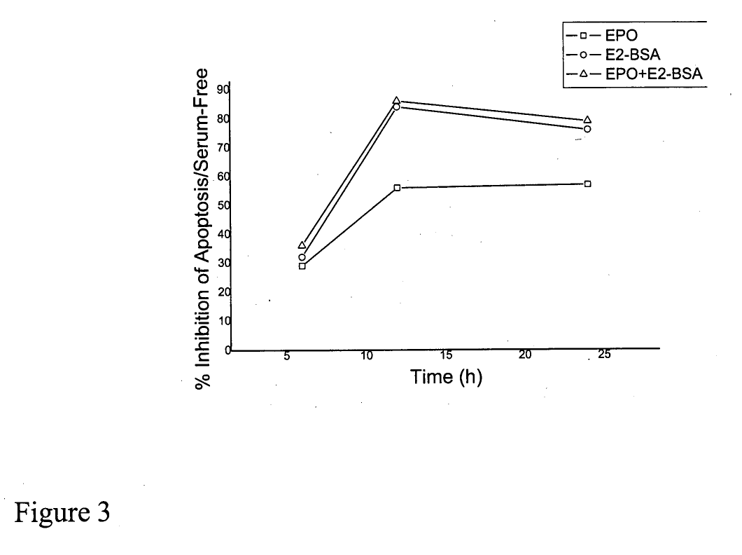

FIG. 3 presents the surprising additive effect of EPO and E2-BSA on the apoptosis of T47D epithelial cells. The effect is apparent only after long incubation times (24h).

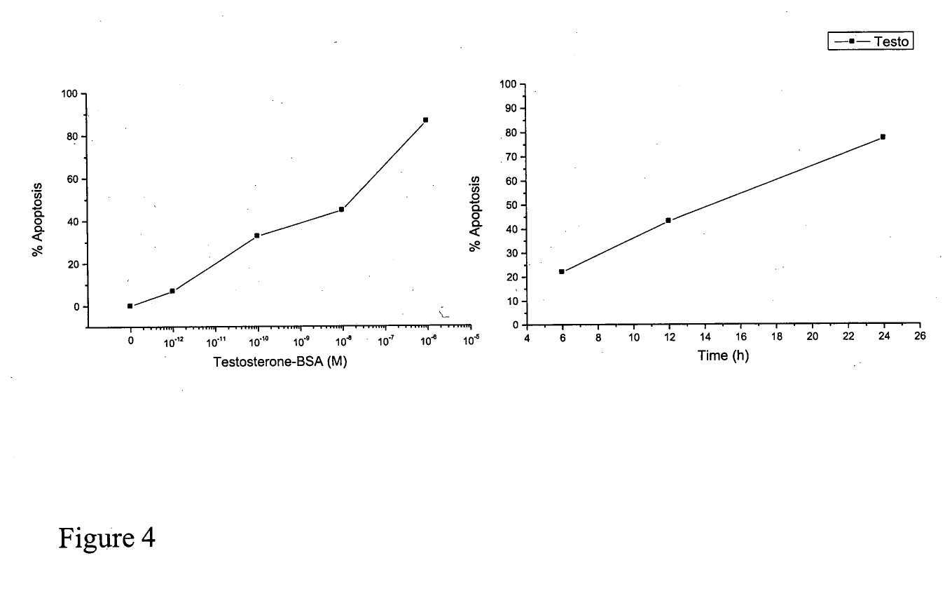

FIG. 4 presents the dose- (left panel) and time-effect of Testosterone-BSA on the apoptosis of T47D breast epithelial cells.

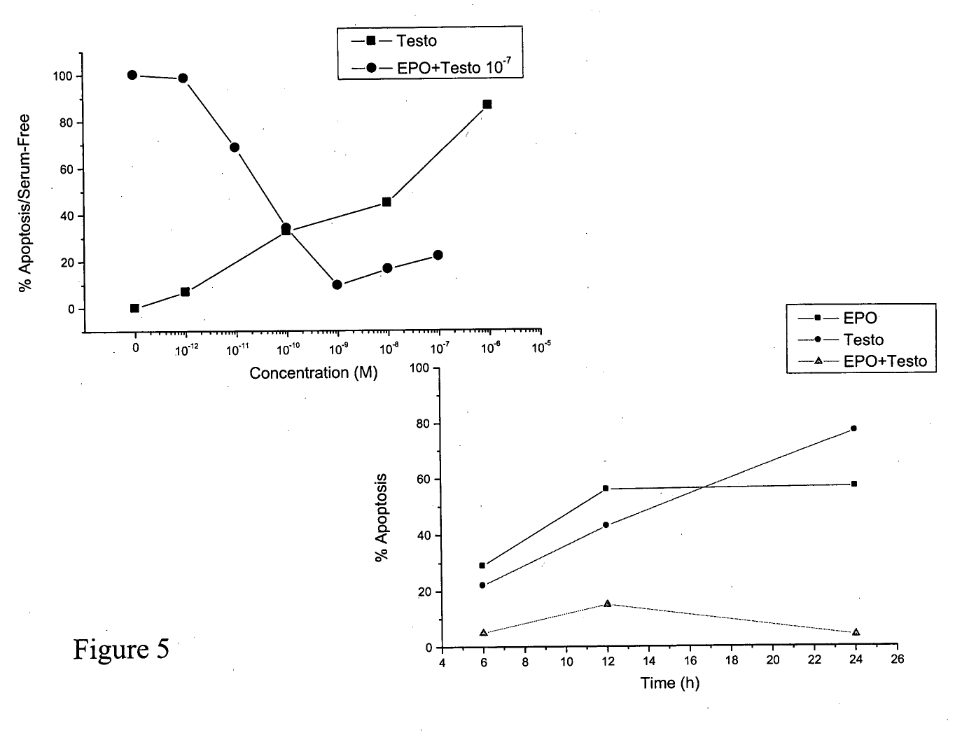

FIG. 5 presents the surprising additive effect of EPO and Testosterone-BSA on the apoptosis of T47D breast epithelial cells. The upper panel shows the pro-apoptotic action of testosterone-BSA (squares). In contrast, when cells were incubated with a maximal dose of testosterone-BSA (10⁻⁷M), inducing ˜80% apoptosis, and varying concentrations of EPO (circles) a complete reversion of apoptosis is observed. The lower panel presents the time effect of EPO, testosterone-BSA and their equimolar association (10⁻⁷M). As shown, the addition of testosterone-BSA, surprisingly potentiates the anti-apoptotic effect of erythropoietin, even after a 6-hour incubation.

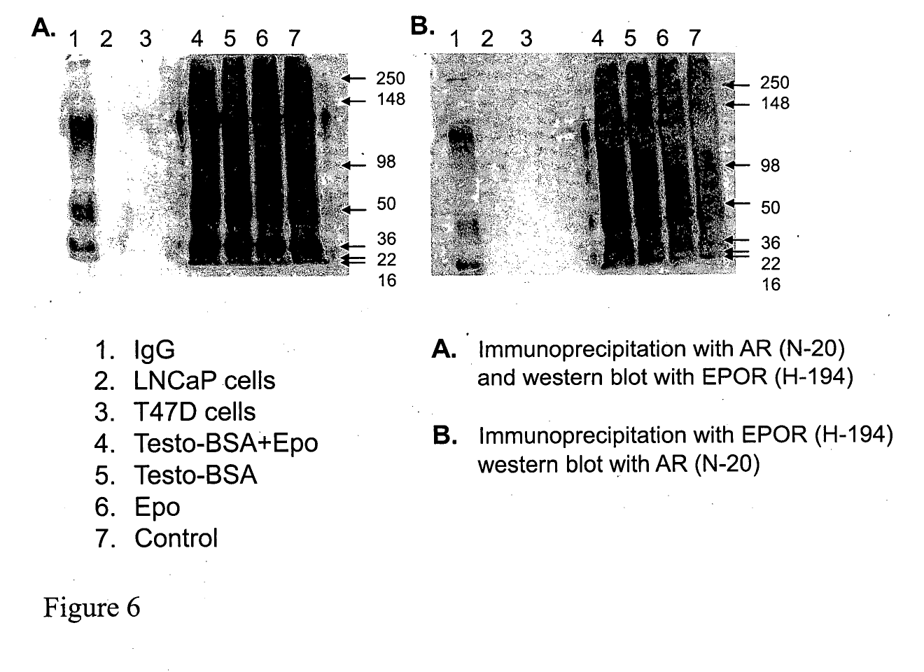

FIG. 6 presents the effect of co-immunoprecipitation and blotting of T47D cell membranes, preincubated either with EPO or testosterone-BSA and immunoprecipitated either with an anti-androgen receptor (AR) antibody and blotted with an antibody against the EPOR (A), or, conversely, immunoprecipitated with an anti-EPOR antibody and blotted with an anti-AR antibody against the amino-terminal of the protein (B). Our previous results on the same cell line (Kampa M, et al Exp Cell Res 2005, 307, 41-51) have shown that membrane androgen receptors can be recognized by the antibody used here. As shown in the figure, no blotting cross-reaction between EPOR (molecular mass 66 kD) and AR (molecular mass 110 kD) is observed.

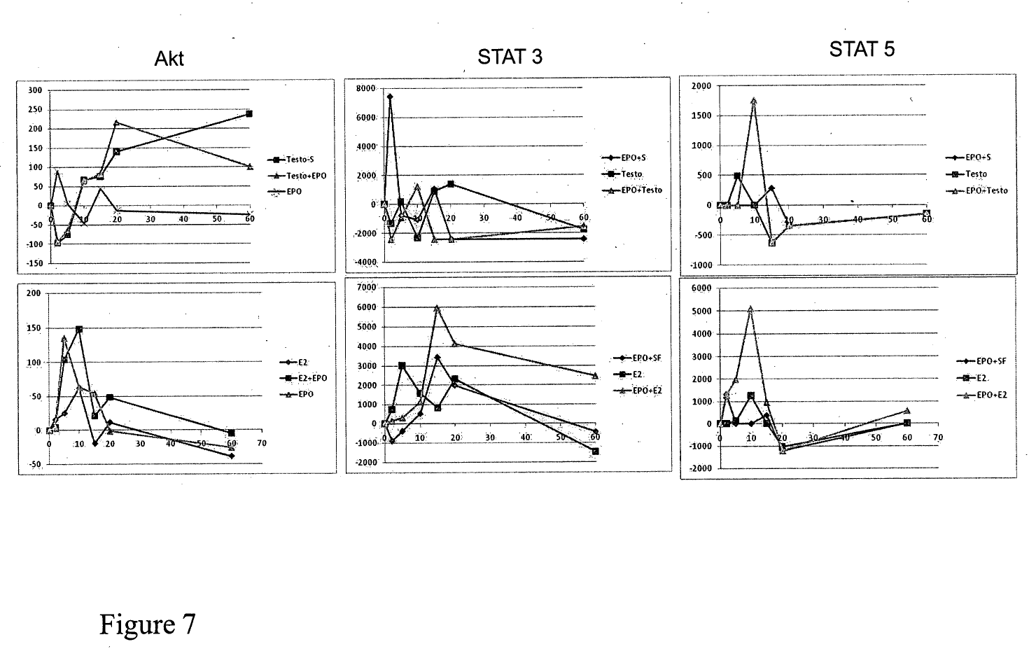

FIG. 7 presents some of the intracellular signaling molecules measured with a multiplex technique. Upper lane shows changes observed in the presence of EPO and/or testosterone-BSA (10⁻⁷M) in the presence of serum, while the lower lane shows the changes in the presence of EPO and/or estradiol-BSA (10⁻⁷M) in the absence of serum.

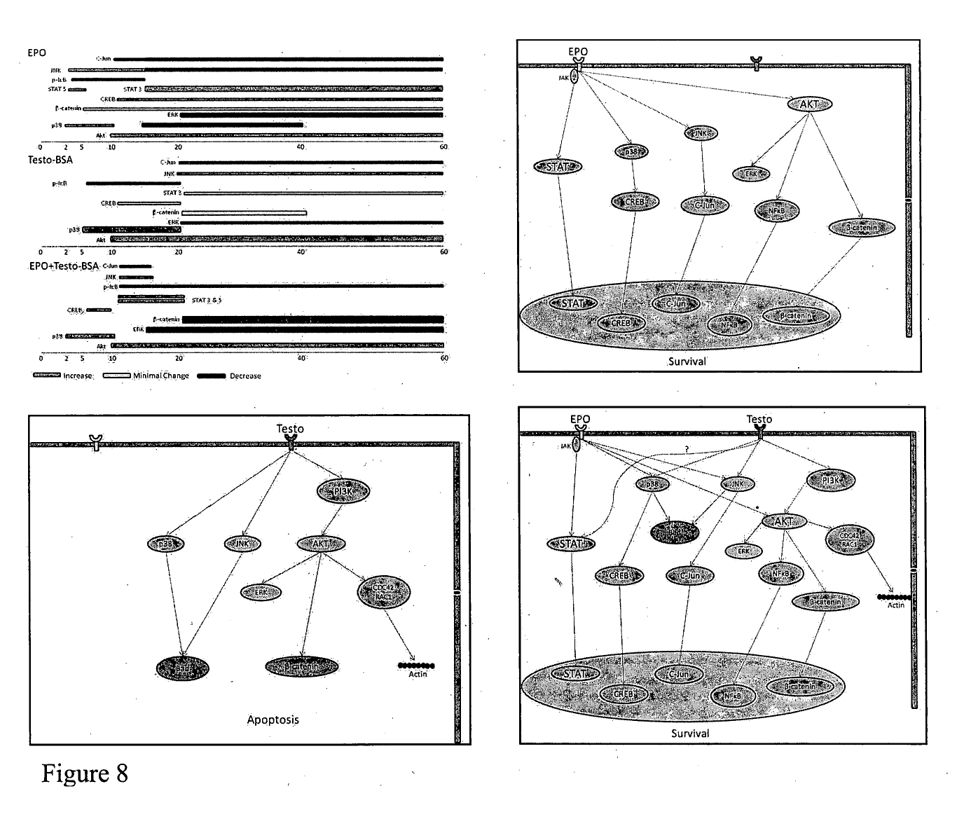

FIG. 8 presents (in the upper left panel) changes in a number of signaling molecules, after the addition of EPO, Testosterone-BSA or their association. The thickness of the bar indicates the relative stimulation (“increase”) or inhibition (“decrease”) of the corresponding molecule. The other three graphs indicate signaling cascades involved in the pro- (testosterone) or anti-apoptotic action of EPO or its association with Testosterone-BSA. It is interesting to note the switch of p38 and Jnk cascade from a pro- to an antiapoptotic action.

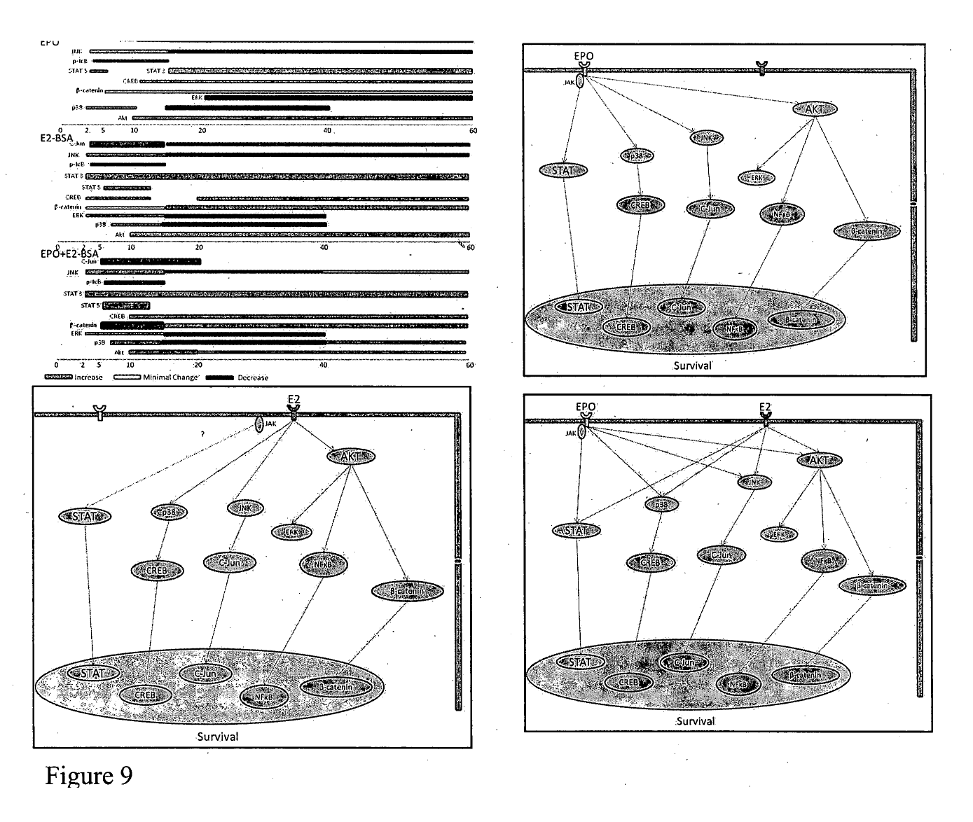

FIG. 9 presents (in the upper left panel) changes in a number of signaling molecules, after the addition of EPO, E2-BSA or their association. The thickness of the bar indicates the relative stimulation (“increase”) or inhibition (“decrease”) of the corresponding molecule. The other three graphs indicate signaling cascades involved in the anti-apoptotic action of EPO, E2-BSA or their association.

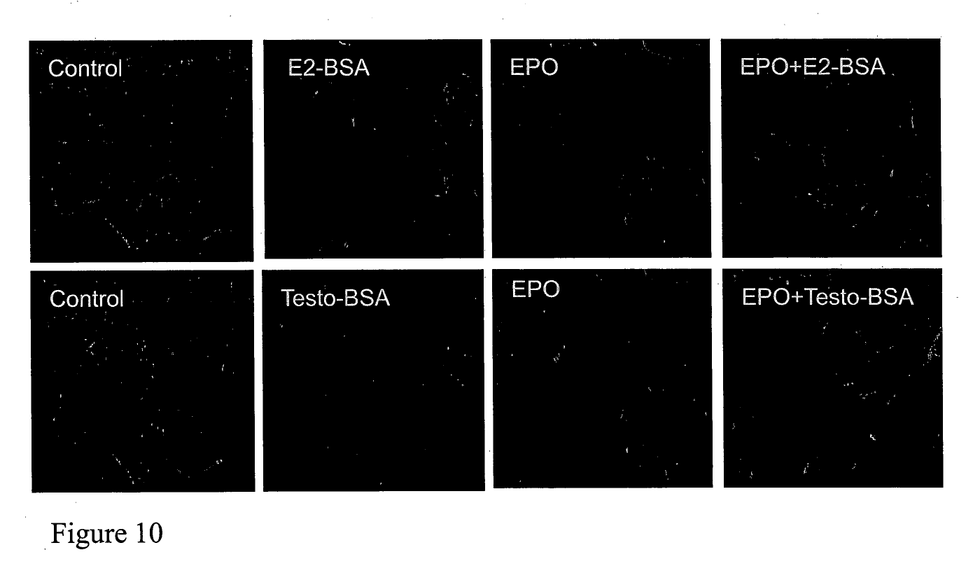

FIG. 10 shows the modification of the actin cytoskeleton after incubation with EPO, E2-BSA, and their association (upper lane), EPO, testosterone-BSA and their association (lower lane). Note the surprising protrusion of filopodia and the formation of lamelipodia after EPO and especially after testosterone-BSA and EPO.

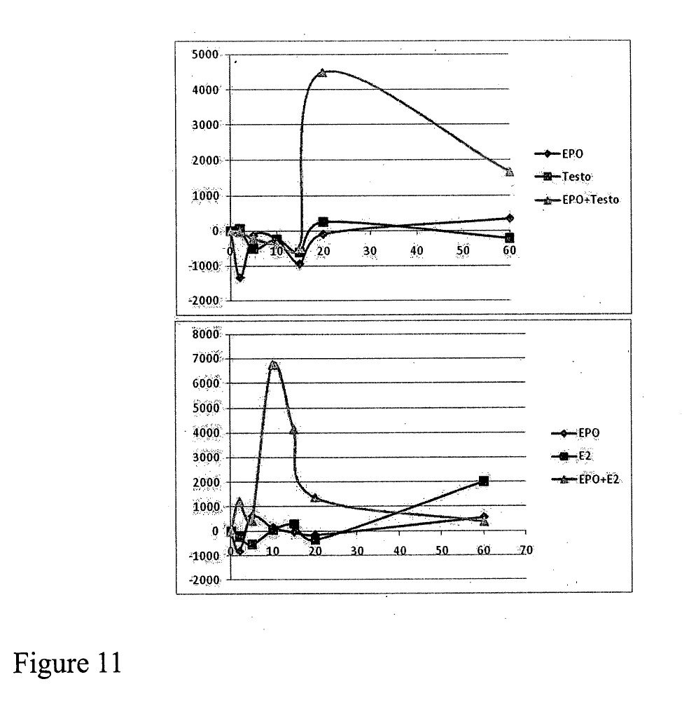

FIG. 11 presents the modification of beta-catenin in T47D cells, incubated in the presence (upper panel) or in the absence (lower panel) of serum, after incubation with EPO, testosterone-BSA (10⁻⁷M) or their equimolar combination (upper panel) or incubation with EPO or estradiol-BSA (10⁻⁷M) or their equimolar combination (lower panel).

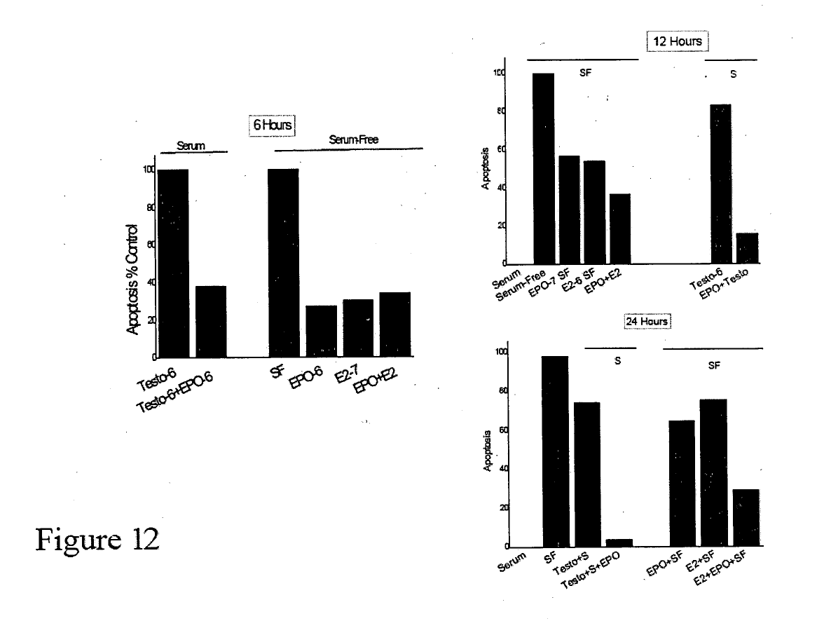

FIG. 12 presents the surprising greater than additive effect of EPO and E2-BSA on the apoptosis of T47D epithelial cells. The effect is apparent after 12, and especially after 24 hours. FIG. 12 also presents the surprising greater than additive effect of EPO and Testosterone-BSA on the apoptosis of T47D breast epithelial cells, as compared to EPO alone. Apoptosis was completely abolished after 24 hours.

### EXAMPLES

### Example 1

Human epithelial cells (for the present examples the human breast epithelial cell line T47D has been used, expressing both estrogen and progesterone intracellular, estrogen and androgen membrane and EPO receptors, see Arcasoy M O, et al Biochem Biophys Res Commun 2003; 307:999-1007; Kampa M, et al Exp Cell Res 2005; 307:41-51) were cultured in serum-supplemented culture medium. Then they were washed, transferred into a serum-free medium, supplemented with the indicated concentrations of EPO (ranging from 10⁻¹² to 10⁻⁷M). Six, twelve and 24 hours later, apoptosis was assayed by the ApoPercentage assay (Biocolor Ltd., Belfast, N. Ireland), detecting initial damage of the plasma membrane of early apoptotic cells. Serum deprivation of cells, through omission of growth factors and/or nutrients, is a major pro-apoptotic challenge. Indeed, as early as 6 hours thereafter, cells enter into apoptosis. However, EPO can partially reverse this apoptotic phenomenon. FIG. 1 presents the dose- and time-dependent inhibition of apoptosis of cells, initiated by EPO.

### Example 2

Human epithelial T47D cells were cultured in the presence of serum. Then they were transferred into a serum-free medium, supplemented with the indicated concentrations of estradiol-BSA conjugate (Sigma-Hellas, Athens, Greece). Apoptosis, measured as indicated in Example 1, was measured after 6, 12 and 24 hours. As shown in FIG. 2, E2-BSA partially reverses serum-deprived-induced apoptosis, in a time- and dose-dependent manner.

### Example 3

Human epithelial T47D cells were cultured, transferred into serum-free medium, supplemented with EPO (10⁻⁷M) and E2-BSA (10⁻⁷M). Six, twelve and 24 hours later, apoptosis was assayed, as described in Example 1. In 6 and 12 hours, the effect of the combination of the agents was equal to each one of them, administered alone. Surprisingly, 24 hours later, an additive effect of E2-BSA and EPO is observed, suggesting a physical or functional interaction of the agents. This interaction may occur at the receptor level (i.e. at the plasma membrane level) or by modifying post-receptor intracellular signaling cascades, leading ultimately to anti-apoptosis. FIG. 3 presents this surprising additive action of E2-BSA and EPO.

### Example 4

Human epithelial T47D cells were cultured in serum supplemented medium. Then, testosterone-BSA (Sigma-Hellas, Athens, Greece) was added (10⁻¹²-10⁻⁶M) and apoptosis was assayed, as detailed in Example 1. As early as 6 hours later, apoptosis can be detected, initiated by testosterone-BSA, in a dose-dependent manner. FIG. 4 shows this well documented apoptosis (see Kampa M, et al Exp Cell Res 2005; 307:41-51). Surprisingly, addition of a maximal dose of testosterone-BSA (10⁻⁶M) with variable doses of EPO (10⁻¹²-10⁻⁷M) reverses completely the apoptotic effect of testosterone. This is also presented in FIG. 5. It is noteworthy that EPO, in a serum-supplemented medium, has no effect at the time examined. This surprising effect of EPO is present not only at 6 hours, but also at 12 and 24 hours (FIG. 5). The interaction of EPO and testosterone-BSA may occur at the receptor level (cross-interaction of either ligand with heterologous receptor, for example interaction of EPO with membrane androgen receptors or vice versa), cross-interaction of EPOR with membrane androgen receptors, or an interaction at a post-receptor level, through modifications of intracellular signaling cascades. The first option (cross-binding of EPO on membrane androgen receptors or testosterone-BSA on EPOR) is not probable. Indeed, flow cytometry analysis performed in T47D cells, labeled with testosterone-BSA-FITC (Sigma-Hellas, Athens, Greece) in the absence or the presence of EPO, shows an equal binding of testosterone. This result indicates that EPO and testosterone-BSA do not bind to the same receptor molecule.

### Example 5

In order to identify a possible interaction of EPOR and membrane androgen receptors, cells were incubated with testosterone-BSA (10⁻⁷M), EPO (10⁻⁷M) or their equimolar combination (10⁻⁷M of each agent). Half an hour later, the medium was eliminated, and cells were washed, homogenized and solubilized, as described in Bonaccorsi L et al, Steroids, 69, 549-52, 2004 and incubated with antibodies against EPOR or androgen receptors (both from Santa Cruz Biotechnology, CA). The potential receptor-antigen complex was absorbed for 12 hours on protein-G-sepharose (Sigma Hellas, Athens, Greece), centrifuged and subjected to polyacrylamide gel electrophoresis, followed by blotting on nitrocellulose membranes. The membranes were then blotted with the other antibody (i.e. cell membranes incubated with anti-EPOR antibody were blotted with anti-AR, while membranes incubated with anti-AR were blotted with an anti-EPOR antibody). As shown in FIG. 6, no cross-reaction between the two receptors was observed, indicating that possibly no hetero-dimers of EPOR-mAR were formed at the cellular membrane level. The above result indicates that the competitive action of EPO and androgens is not mediated at the cellular membrane level, but is due to a possible interaction at the signal transduction level.

### Example 6

T47D cells were incubated in the presence of EPO (10⁻⁷M), testosterone- or estradiol-BSA (10⁻⁷M) or their equimolar combination (10⁻⁷ M each). At the corresponding time points, as described in FIG. 7, cells were lyzed in lysis buffer, centrifuged, and the supernatant was analyzed for the presence of phosphorylated or non-signaling molecules, as shown in the figure, using a multiplex signaling assay from Upstate (Lake Placid, N.Y.). The Figure presents the activation of each signaling molecule with time, in the presence of each agent or their combination.

Based on the above results, FIGS. 8 and 9 present schematically activated or inhibited signaling molecules (upper left panels) and signal transduction pathways after activation of EPOR and mAR (FIG. 8) and EPOR and mER (FIG. 9). Apoptotic and survival pathways are presented.

### Example 7

A known element implicated in cytokinesis and the fate of the cell is cortical actin. Actin bundles, through a dynamic interaction into the cell and by regulating the cell-substratum interactions, control a number of phenomena, from cell motility (initiated and sustained by fillopodia and lamelipodia), cell division, and cell fate, by initiating apoptosis or anoikis, depending on whether it occurs through intracellular stimuli or the interaction of the cell with its substratum, respectively. Testosterone-BSA (as well as E2-BSA) is a known modifier of cortical actin polymerization state (see Papakonstanti E A, et al Mol Endocrinol 2003; 17:870-81; Kampa M, and Castanas E. Mol Cell Endocrinol 2006; 246:76-82). Surprisingly, actin dynamics were also modified by EPO, with the formation of lamelipodia. Even more surprisingly, when epithelial cells were incubated with EPO and testosterone, a profound modification of cortical actin was observed, with massive formation of filopodia and lamelipodia. These examples are presented in FIG. 10. The modifications of cortical and intracellular actin are directly related to cell proliferation, maturation and migration. This actin modification under the combined action of EPO with testosterone- or estradiol-BSA is followed by a modification of beta-catenin (FIG. 11), indicating an implication of these molecules in cytokinesis. In this respect and under the combined action of testosterone-BSA and EPO, it is believed that this association could be of value for tissue regeneration, wound healing and other medical and cosmetic applications.

### Example 8

Cells (T47D human breast cancer epithelial cell line) were incubated in serum supplemented (S) or serum-free (SF) culture medium, for the indicated time-periods, in the absence or the presence of EPO or E2-BSA (E2) or Testosterone-BSA (Testo) and their combination. EPO, E2-BSA and Testosterone-BSA were all used at a concentration of 10⁻⁷M. Apoptosis was assayed with the BioColor ApoPercentage kit as described in Example 1.

The results showed that EPO was inactive in serum-supplemented medium (data not shown). However, in the absence of serum, it decreased significantly apoptosis induced by the absence of serum. This effect was more pronounced at short (6 hours) as compared to longer incubation time 12 or 24 hours), attributed to the progressive degradation of the peptide. The same effect was observed under the action of E2-BSA. In contrast, the simultaneous addition of E2-BSA and EPO induced a more-than additive sustained and time-dependent inhibition of apoptosis, obvious at 12 and especially 24 hours.

Testosterone-BSA, as reported previously, induced an induction of apoptosis in serum-supplemented medium. The addition of EPO to the cultures (which alone had no effect on apoptosis) induced a time-dependent reduction of apoptosis. Apoptosis was completely abolished after 24 hours.

The conclusion which can be drawn from these experiments is that the addition of steroid membrane receptor agonists potentiates and prolongs the anti-apoptotic action of EPO.

(The above Examples use a breast, cancer model which is a good general model of the behaviour of cells, in particular epithelial cells and solid epithelial tumors, as well as breast epithelial cells and breast tumors).

1. A composition comprising (i) a membrane steroid receptor agonist and (ii) erythropoietin (EPO).

2. The composition of claim 1, wherein the membrane steroid receptor agonist is selected from the group consisting of glucocorticoid, mineralocorticoid, estrogen, progestin, progesterone, androgen and vitamin-D receptor agonists.

3. (canceled)

4. The composition of claim 1, wherein the membrane steroid receptor agonist is a steroid conjugate.

5. The composition of claim 1, wherein the membrane steroid receptor agonist is a macromolecular conjugate of a steroid with a protein or peptide.

6. The composition of claim 5, wherein said protein is a globular protein, a plasma protein, an albumin or an antibody.

7. The composition of claim 6, wherein said albumin is human serum albumin, bovine serum albumin or albumin from another species.

8. The composition of claim 4, wherein said steroid in the conjugate is selected from the group consisting of glucocorticoids, mineralocorticoids, estrogens, progestins, progesterone, androgens and vitamin-D.

9. The composition of claim 8, wherein said steroid is an estrogen or an androgen.

10. The composition of claim 9, wherein said steroid is testosterone or estradiol.

11. The composition of claim 1, wherein the membrane steroid receptor agonist comprises a micro- or macromolecular natural or synthetic agent, with or without a cholesterol skeleton.

12. The composition of claim 11, wherein said membrane steroid receptor agonist is a micromolecular agonist selected from the group consisting of natural or modified catechins, epicatechins and proanthocyanidolic condensed tannins.

13. The composition of claim 1, wherein said membrane steroid receptor agonist comprises an antibody.

14-15. (canceled)

16. A kit comprising (i) a membrane steroid receptor agonist and (ii) erythropoietin (EPO).

17. The kit of claim 16, wherein said membrane steroid receptor agonist is selected from the group consisting of glucocorticoid, mineralocorticoid, estrogen, progestin, progesterone, androgen and vitamin-D receptor agonists.

18-19. (canceled)

20. The composition of claim 1, wherein EPO has one or more actions selected from the group consisting of trophic, regenerative, proliferative and anti-apoptotic actions.

21-23. (canceled)

24. A method of potentiating the action of EPO in a subject, which method comprises administering to a subject in need thereof a therapeutically effective amount of a membrane steroid receptor agonist and EPO.

25. The method of claim 24, wherein said action of EPO is selected from the group consisting of a trophic, a regenerative, a proliferative and an anti-apoptotic action.

26. The method of claim 24, wherein said membrane steroid receptor agonist is selected from the group consisting of glucocorticoid, mineralocorticoid, estrogen, progestin, progesterone, androgen and vitamin-D receptor antagonists.

27. A method of altering an activity of a cell, comprising contacting a cell with an effective amount of a membrane steroid receptor agonist and EPO, wherein the activity of the cell is one or more activities selected from the group consisting of apoptosis, proliferation, differentiation, migration and regeneration.

28. (canceled)

29. A method of altering an activity of a cell in a subject, comprising administering to a subject in need thereof an effective amount of a membrane steroid receptor agonist and EPO, wherein the activity of the cell is one or more activities selected from the group consisting of apoptosis, proliferation, differentiation, migration and regeneration.

30. The method of claim 27, wherein the activity of the cell is apoptosis.

31. The method of claim 30, wherein said method reduces apoptosis.

32. The method of claim 27, wherein said membrane steroid receptor agonist is selected from the group consisting of glucocorticoid mineralocorticoid, estrogen, progestin, progesterone, androgen and vitamin-D receptor agonists.

33. (canceled)

34. The method of claim 27, wherein the cell is at least one of an epithelial cell, an endothelial cell, a mesenchymal cell and a cell of a bone marrow lineage.

35. (canceled)

36. The method of claim 34, wherein said cell is a cell of a bone marrow lineage, and wherein the bone marrow lineage is one or more selected from the group consisting of erythoid, lymphoid, mononuclear and granulocytic cells.

37. The method of claim 29, wherein the subject is a human.

38. The method of claim 29, wherein said method further induces wound healing, regeneration or protection of organs, tissues, or cells, or is an anti-apoptotic therapy.

39. The method of claim 29, wherein said method further treats anemia, heart failure, brain injury, stroke, wounds, or a disease in which regeneration of cells, tissues or organs is required.

40. The composition of claim 11, wherein the membrane steroid receptor agonist is a steroid or a phenolic compound

41. A pharmaceutical composition comprising the composition of claim 1 and a pharmaceutically acceptable carrier or diluent.

42. The method of claim 24, wherein the subject is a human.

43. The method of claim 24, wherein said method further induces wound healing, regeneration or protection of organs, tissues, or cells, or is an anti-apoptotic therapy.

44. The method of claim 24, wherein said method further treats anemia, heart failure, brain injury, stroke, wounds, or a disease in which regeneration of cells, tissues or organs is required.

45. The method of claim 24, wherein the membrane steroid receptor agonist and EPO are administered simultaneous, separately or sequentially.

46. The method of claim 29, wherein the activity of the cell is apoptosis.

47. The method of claim 46, wherein said method reduces apoptosis.

48. The method of claim 29, wherein said membrane steroid receptor agonist is selected from the group consisting of glucocorticoid, mineralocorticoid, estrogen, progestin, progesterone, androgen and vitamin-D receptor agonists.

49. The method of claim 29, wherein the cell is at least one of an epithelial cell, an endothelial cell, a mesenchymal cell and a cell of a bone marrow lineage.

50. The method of claim 49, wherein said cell is a cell of a bone marrow lineage, and wherein the bone marrow lineage is one or more selected from the group consisting of erythoid, lymphoid, mononuclear and granulocytic cells.
